(19) United States
(12) Patent Application Publication
(10) Pub. No.: US 2026/0000504 A1
(43) Pub. Date: Jan. 1, 2026

(54) CARDIAC VALVE WITH EARLY PREFERENTIAL POLARIZATION TOWARDS AN M2 PHENOTYPE POST-IMPLANTATION, AND METHOD OF IMPLANTATION

(71) Applicant: DiFusion, Inc., Austin, TX (US)

(72) Inventors: Derrick Johns, Liberty Hill, TX (US); Joseph J. Crudden, Hudson, NH (US)

(21) Appl. No.: 18/880,929

(22) PCT Filed: Jun. 27, 2023
(86) PCT No.: PCT/US2023/026337

§ 371 Date: Jan. 3, 2025

Related U.S. Application Data
(60) Provisional application No. 63/358,394, filed on Jul. 5, 2022.

(51) Int. Cl. A61F 2/24; A61L 27/44

(52) U.S. Cl. CPC A61F 2/24; A61L 27/446; A61F 2210/0071; A61F 2250/0067; A61L 2300/25; A61L 2400/18; A61L 2430/20

(57) ABSTRACT

Cardiac replacement valves that are anti-biofilm and tissue-integrating implantable biomaterial devices that optionally can elute therapeutic ions such as magnesium, silver, copper and/or zinc. In certain embodiments, the devices are hydrophilic due to the presence of ceramic particles such as zeolite. In certain embodiments, the devices are subjected to a surface treatment such as a plasma treatment or a corona discharge treatment to enhance the immobilization of integrin-stimulating peptides such as RGD to the biomaterial for cell adhesion enhancement when implanted in a host. Methods of replacing diseased valves in a patient are also disclosed.

This application claims priority of U.S. Provisional Application Ser. No. 63/358,394 filed Jul. 5, 2022, the disclosure of which is incorporated herein by reference in its entirety.

### BACKGROUND

Biomaterials may be surgically implanted into the body for various reasons, including orthopedic applications (e.g., hip replacement, skull flaps, dental implants, spinal procedures, knee replacement, bone fracture repair, etc.), surgical repair applications (e. g., ACL screws, surgical meshes, etc.), heart valve repair or replacement, and others. In view of the structural integrity required by many such devices, particularly those involving bone repair or replacement, and biocompatibility requirements, materials of fabrication are limited and generally consist of metal, plastic and composites. Each has its advantages and disadvantages.

Benefits derived from these devices are often offset by infection which in some cases can lead to sepsis and death of the host. The most common organisms causing infections are Staphylococcus epidermidis and Staphylococcus aureus. Staphylococcus epidermidis is a major component of the normal bacterial flora of human skin and mucous membranes. It is a common pathogen that often colonizes patients in hospital settings who have surgical implants due to the microbes' ability to adhere to medical devices and form a biofilm. Additionally, methicillin-resistant Staphylococcus aureus (MRSA) is a type of Staphylococcus bacteria that is resistant to many antibiotics is therefore of particular concern. Other gram-positive bacteria, gram-negative bacteria and fungal organisms also are causative organisms that may be problematic. As microorganisms come in close proximity to the surface of the medical device, they will either be attracted or repelled by it depending on the sum of the different non-specific interactions. In biological systems, hydrophobic/hydrophilic interactions play an important role in the pathogenesis of a wide range of microbial infections.

Certain polymeric materials, such as polyetherketoneketone (PEKK) and polyetheretherketone (PEEK), have been found to be a useful material for medical implants. A suitable polymeric material for implant applications should interact well with tissue, and also should be recognized by the host as natural so as to minimize or avoid becoming encapsulated by a fibrous apposition layer of soft tissue.

The role of the neutralization of infectious agents, and the host response to foreign materials such as surgical implants in normal tissue/organ development and tissue regeneration, is important. Immune cells such as neutrophils, macrophages, and lymphocytes possess robust plasticity with respect to phenotype. For example, macrophages typically show a marked pro-inflammatory (M1-like) phenotype when presented with certain antigens (e.g., synthetic foreign materials or bacteria), but then transition to pro-healing, anti-inflammatory and constructive phenotype (M2-like) when subsequently influenced by alternative signaling molecules. A “normal” response to injury involves an initial pro-inflammatory cell response that must then transition to a pro-healing phenotype lest there be continuous, non-healing inflammation and tissue destruction. The phenotype of cells such as macrophages can be determined, at least in part, by the expression of certain markers that are detected by immunolabeling. Macrophage phenotype during the early response (i.e., 7-14 days) to an implanted foreign material is predictive of the downstream outcome. An early M1-like response has been associated with chronic inflammation and fibrosis; whereas an early M2-like response has been associated with minimal fibrosis and constructive and functional tissue remodeling.

It is therefore important that implantable biomaterials be developed that promote activation of one or more genes associated with an M2-like macrophage phenotype. A desirable M1/M2 macrophage phenotype balance, and in particular, the early preferential polarization towards an M2 phenotype after implantation, can lead to a shorter pro-inflammatory period and earlier reparative process, which can be critical for effective tissue integration and ultimately implant success.

Cardiac valvular dysfunction such as regurgitation or valve insufficiency places undue strain on the heart, and eventually can result in morbidity and mortality. A feared complication of artificial valvular disease is stroke. Embolic stroke and artificial valvular insufficiency can result from reactive material adhering to the surface of the valvular leaflets. Aortic, mitral and tricuspid valve disease is typically treated by either surgical repair, such as with an annuloplasty ring or surgical replacement with a valve prosthesis. Such surgery generally requires the use of a heart-lung machine for circulation of the blood as the heart is stopped and then opened during the surgical procedure, where the cardiac valve prosthesis and/or annuloplasty rings are implanted such as by suturing.

The promotion of activation of one or more genes associated with an M2-like macrophage phenotype, and a desirable M1/M2 macrophage phenotype balance is of particular importance where the implant is a heart valve or heart valve component, as chronic inflammation is a well-documented problem with such valves, particularly aortic valves, regardless of whether the implant is a biological valvular prosthesis or a mechanical one. Indeed, in one study, approximately 50% of patients continued to have a post-operative active systemic inflammatory state that was even higher than the preoperative inflammatory state.

In addition, as microorganisms come in close proximity to the surface of the device, they will either be attracted or repelled by it depending on the sum of the different non-specific interactions. In biological systems, hydrophobic/hydrophilic interactions play an important role in the pathogenesis of a wide range of microbial infections. Many resins including polyetheretherketone (PEEK) are hydrophobic materials and bacteria tend to adhere easily to these types of surfaces. They are also organic materials which do not carry significant surface charges. Consequently, it would be desirable to develop a cardiac implant or device that has reduced hydrophobic properties, and/or that has a net negative charge, particularly at an exposed surface when implanted into a host.

The addition of ceramics such as zeolites to such resins helps accomplish this. For example, zeolite may be incorporated into the resin to create a composite material with ceramic character that confers charge to the surface and renders it hydrophilic. In addition, zeolites can provide ion-exchange sites which optionally can be loaded with one or more therapeutic metal ions that elute when in contact with the bodily fluid or tissue of a host, thereby imparting therapeutic activity to the implant site, such as antimicrobial activity and/or anti-coagulating activity. However, poorly controlled release of metal ions because of device design can result in the deleterious accumulation of excess metal ions (e.g., silver, copper) in the host over time. Carefully controlled release of therapeutic metal ions, from ion exchange ceramics such as zeolites, incorporated into polymer composites which are used to fabricate medical devices can provide for precision controlled release of the correct, safe and efficacious level of therapeutic ion(s). Accordingly, embodiments disclosed herein relate to implantable medical devices such as heart valves or heart valve components that are composed of, and/or are coated with, and/or contain, one or more resins, wherein the resins include a favorable immune response modulating agent, wherein the modulating agent is a ceramic such as an aluminosilicate, preferably zeolite, and the immune response that is modulated is an inflammatory response. Embodiments disclosed herein also relate to implantable medical devices such as heart valves or heart valve components where the valve includes a fabric or textile that is sutured at a target implant site in the heart of a host, and the fabric or textile is modified to include a favorable immune response modulating agent, wherein the modulating agent is a ceramic such as an aluminosilicate, preferably zeolite, and the immune response that is modulated is an inflammatory response. The ceramic present in the device, whether it be in the valve body resin or the fabric or both, may optionally be loaded with one or more ion-exchangeable cations. The one or more cations may elute from the resin, fabric and/or textile upon exposure to bodily fluid, and provide a therapeutic effect to the host, such as antimicrobial and/or anticoagulative activity.

Efforts to enhance tissue integration are ongoing, including via functionalizing biomaterial surfaces with integrating agents. The arginine-glycine-aspartic acid sequence (RGD tripeptide sequence) is present in many proteins that function in cell adhesion, such as fibronectin, vitronectin, osteopontin, fibrinogen and collagen. This small peptide has a high affinity for one or more integrins and as such, may be characterized as an integrin-stimulating peptide, and can promote increased binding of osteogenic cells to biomaterials, as integrin-RGD peptide binding plays an important role in cell growth, migration and survival. Integrins are heterodimeric cell surface receptors that mediate adhesion between cells and the extracellular matrix by binding to ligands having an exposed RGD sequence. Peptides which contain this sequence can mimic the ligands of certain integrins and bind to them.

RGD is highly effective at promoting the attachment of numerous cell types to a plethora of diverse materials. RGD is the principal integrin-binding domain present within ECM proteins such as fibronectin, vitronectin, fibrinogen, osteopontin, and bone sialoprotein. RGD is also present in some laminins and collagens, however RGD may be inaccessible within these molecules (depending upon conformation), and other amino acid motifs are known to serve as alternative binding modules for laminin and collagen-selective receptors. The RGD sequence can bind to multiple integrin species, and synthetic RGD peptides offer several advantages for biomaterials applications. Because integrin receptors recognize RGD as a primary sequence (although conformation of the peptide can modulate affinity), the functionality of RGD is usually maintained throughout the processing and sterilization steps required for biomaterials synthesis, many of which cause protein denaturation. The use of RGD, as compared with native ECM proteins, also minimizes the risk of immune reactivity or pathogen transfer, particularly when xenograft or cadaveric protein sources are utilized. Another benefit is that the synthesis of RGD peptides is relatively simple and inexpensive, which facilitates translation into the clinic. Further still, RGD peptides can be coupled to material surfaces in controlled densities and orientations. These advantages of straightforward synthesis, minimal cost, and tight control over ligand presentation cannot readily be achieved when using full-length native matrix proteins to functionalize material surfaces.

However, difficulties arise in binding RGD and other peptides to resins in an integrin-stimulating effective amount.

It therefore would be desirable to effectively functionalize implant material surfaces with peptide ligands, including biomimetic peptides, such as those comprising the RGD sequence in an effort to stimulate and/or enhance tissue integration of the implant in a host. It further would be desirable to enhance the implant life in a host by increasing the adhesion between natural tissue and the synthetic implant material, which will lead to an improved host response, including faster host recovery time, lower need for repeated surgical intervention and lower medical costs. Decreased reoperations is important given the higher complication rates of repeated procedures.

Accordingly, embodiments disclosed herein also relate to implantable heart valves that are composed of, or coated with, or contain, one or more resins, wherein at least a surface of the resin has a tissue integrating effective amount of one or more integrin-stimulating peptides bound thereto or immobilized thereon. Integrins stimulate cell adhesion. In some embodiments, the effective amount is an amount sufficient to cause at least 25% enhancement in tissue attachment to the implant compared to a control. The integrin-stimulating peptides may be present in addition to the favorable immune response modulating agent, or instead of it,

Embodiments disclosed herein also relate to methods of manufacturing such implantable heart valves, methods of altering one or more surface properties of such devices and immobilizing integrin-stimulating peptides on one or more surfaces of such devices, such as peptides of the tripeptide motif RGD, and methods of implanting such devices.

### SUMMARY

The shortcomings of the prior art have been overcome by embodiments disclosed herein, which include engineered implantable biomaterial devices that optionally can elute therapeutic ions. In certain embodiments, the implants are cardiac replacement valves, such as replacement aortic or mitral valves, or components thereof, and are so shaped or configured. In some embodiments, the devices are composed of, contain or are coated with a polymer, such as a polyarylether ketone such as polyetheretherketone (PEEK). In some embodiments, the polymer includes a ceramic material, preferably a zeolite, and the ceramic material optionally may be loaded with one or more therapeutic metal ions, such as silver, copper and/or zinc, that exhibit a therapeutic effect such as antimicrobial or anticoagulative properties when implanted into a body and exposed to bodily fluid or tissue. The devices, when implanted into a body and exposed to bodily fluid, may elute metal ions in a therapeutically effective amount. In certain embodiments, disclosed are methods of imparting therapeutic activity to devices by controlling the delivery of certain cations through exchange via a ceramic material, preferably zeolite, incorporated in the device introduced in a patient. In some embodiments, the ceramic material does not contain a metal ion, yet imparts hydrophilicity and a negative charge to the implant. This helps prevent biofilm formation and enhances cellular adhesion. In embodiments where antimicrobial metal ions are present, the resin/zeolite combination increases the ability of the therapeutic moieties to permeate in and kill the bacterial pathogen rather than be repelled by the hydrophobic surface properties of the naked resin such as PEEK.

In some embodiments, the implants are implantable biomaterial devices having tissue properties. integrating In certain embodiments, disclosed is a biofunctionalized implant having a surface that promotes cell adhesion to the implant. In some embodiments, the implant, or a coating on the implant, is composed of a biomaterial that includes a polymer resin such as PEKK, PEEK, polylactic acid (PLA) and/or polymethyl (meth) acrylate. In embodiments where a coating is applied to a metal body or substrate, such as titanium, the coating may serve as a barrier to the release of metal ions from the metal body that, if released, would lead to inflammation in the host. In certain embodiments, the implant comprises a polymer resin that has been functionalized with a therapeutically effective amount of one or more peptides. Preferably the peptide is an integrin-stimulating peptide and contributes to integrin-ligand binding affinity. Preferably the peptide is peptide that includes the motif RGD, i.e., a peptide that includes the RGD sequence (R=arginine; G=glycine; D=aspartic acid) of amino acids. Most preferably the peptide is linear or cyclic RGD (CRGD).

In certain embodiments, adherence or linking of the one or more peptides to the polymer resin implant base material is enhanced by subjecting the resin to a surface-activating treatment. In various embodiments, the surface-activating treatment includes plasma treatment. In some embodiments, the surface-activating treatment is carried out by exposing the device to plasma, such as with corona discharge. In certain embodiments, the implant, or preferably the plasma-activated implant, is exposed to an activating agent that adds functionality for immobilization of the integrin-stimulating peptide, such as amino functionality, hydroxyl functionality, amide functionality and/or carboxyl functionality. The activating agent enhances the coupling of the integrin-stimulating peptide to the implant.

Disclosed is a cardiac replacement valve assembly comprising a valve body having a passageway through which blood flows when implanted into a host patient, a valve cooperating with the valve body to allow blood flow through the passageway in a first position and block blood flow through the passageway in a second position, and a suture ring extending from the valve body, wherein the valve body comprises a thermoplastic resin having a ceramic material, such as aluminosilicate particles, incorporated therein, the ceramic material being present in the resin in an amount sufficient to impart a negative charge to the exposed surface of the valve assembly and/or render it hydrophilic. The suture ring may comprise fabric, and the fabric may be infused with a ceramic material such as an aluminosilicate, preferably a zeolite,

In some embodiments, the thermoplastic resin comprises polyetheretherketone. In some embodiments, the aluminosilicate is represented by the formula XM_{2/n}O·Al₂O₃·YsiO₂·ZH₂O wherein M represents an ion-exchangeable ion, n represents the atomic valency of the (metal) ion, X and Y represent coefficients of metal oxide and silica respectively, and Z represents the number of water of crystallization. In some embodiments, the valve body has a surface region having been subjected to surface activation and comprising a therapeutically effective amount of an integrin-stimulating peptide immobilized thereon. The surface region may be activated by plasma treatment or corona discharge treatment. The integrin-stimulating peptide may be a peptide containing the amino acid sequence RGD, and may be immobilized on the surface region with an activating agent providing functionality selected from the group consisting of amino functionality, hydroxyl functionality, amide functionality and carboxyl functionality. In some embodiments, the valve may include both the ceramic material and the integrin-stimulating peptide.

Also disclosed is a method of enhancing preferential polarization towards an M2 phenotype post-implantation of a cardiac valve, comprising excising from a patient a cardiac valve, and implanting in the patient a replacement cardiac valve assembly comprising a valve body having a passageway through which blood flows when implanted into the patient, a valve cooperating with the valve body to allow blood flow through the passageway in a first position and block blood flow through the passageway in a second position, and a suture ring extending from the valve body, wherein the valve body comprises a thermoplastic resin having a ceramic material, such as aluminosilicate particles, preferably a zeolite, incorporated therein, the ceramic material being present in the resin in an amount sufficient to impart a negative charge to the exposed surface of the valve assembly, and/or render it hydrophilic.

### BRIEF DESCRIPTION OF THE DRAWINGS

FIG. 1 is a standard curve of fluorescence vs. cell concentration;

FIG. 2 is a bar graph of cell proliferation and attachment in accordance with certain embodiments; and

FIG. 3 is another bar graph of cell proliferation and attachment in accordance with certain embodiments.

### DETAILED DESCRIPTION

Although specific terms are used in the following description for the sake of clarity, these terms are intended to refer only to the particular structure of the embodiments selected for illustration in the drawings. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2% to 10” is inclusive of the endpoints, 2% and 10%, and all the intermediate values).

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that some terms used herein are relative terms. For example, the terms “upper” and “lower” are relative to each other in location, i.e. an upper component is located at a higher elevation than a lower component, and should not be construed as requiring a particular orientation or location of the structure. As a further example, the terms “interior”, “exterior”, “inward”, and “outward” are relative to a center, and should not be construed as requiring a particular orientation or location of the structure.

The terms “top” and “bottom” are relative to an absolute reference, i.e. the surface of the earth. Put another way, a top location is always located at a higher elevation than a bottom location, toward the surface of the earth.

Certain embodiments relate to a biomaterial useful as a cardiac valve or valve component surgical implant comprising a base material such as a polymer resin. Typically the valve functions as a one-way check valve, allowing blood flow in a single direction when the valve is open, and preventing backflow when the valve is closed. Multi-leaflet valves, especially bileaflet valves, are the most commonly used and are suitable, and typically have two semicircular leaflets that pivot on hinges attached to a rigid ring, collar or cuff in response to differential pressures on either side of the valve.

Suitable biomaterials include polyetheretherketone (PEEK), polyetherketoneketone (PEKK), poly(lactic acid) or derivatives thereof (e.g., hydrolyzed or carboxylated poly (lactic acid), or a mixture of the same), or other suitable substrates that may be coated with such a biomaterial, such as titanium, titanium alloys (e.g., nickel titanium, titanium-zirconium-molybdenum (TZM), tungsten-rhenium), stainless steel or alloys of stainless steel, cobalt-chrome, etc. Other suitable resins include thermoplastics, low density polyethylene, polypropylene, ultra-high molecular weight polyethylene or polystyrene, polytetrafluoroethylene (PTFE), expanded PTFE, nylon, polyether-block co-polyamide aliphatic polyether polyurethanes, polyurethane, polymers, polyvinyl chloride, ABS resins, silicones, rubber, polymethylmethacrylate (which melts at about 320° C.) and mixtures thereof, and reinforced resins, such as ceramic or carbon fiber-reinforced resins, particularly carbon fiber-reinforced PEEK. PEEK is particularly preferred, and melts at between 385 and 400 degrees Celsius. In embodiments where the substrate is not a biopolymer, a biopolymer may be incorporated in or on a surface or coated on the substrate.

Accordingly, disclosed are medical implants and methods of their manufacture. In some embodiments, the implant has a main body region and an exposed surface region, the exposed surface region being configured to be exposed to bodily fluid or tissue of a host when the medical implant is implanted in the host. In various embodiments the implant is subjected to surface activation. Surface activation may enhance the binding of one or more therapeutic agents thereto. In some embodiments, the surface activation is carried out by subjecting the implant to plasma treatment. In some embodiments, the surface activated implant may be exposed to an activating agent such as ammonium hydroxide to provide amino functionality to immobilize the therapeutic agent. In some embodiments, the therapeutic agent comprises a peptide. In some embodiments, the peptide is a biomimetic peptide. In certain embodiments, the peptide is linear or cyclic RGD bound to or immobilized on the implant in an amount effective to stimulate osseointegration of the implant when the implant is implanted in a host. In some embodiments, the integrin-stimulating peptide is covalently linked to the implant surface via an N-terminal amino group.

In various embodiments, the implants achieve enhanced tissue adhesion that is at least about 120% greater than adhesion achieved with an otherwise identical implant that has not been subject to surface activation or surface functionalization with an integrin-stimulating peptide (i.e., a control) (hereinafter “the enhanced adhesion amount”). In some embodiments the enhanced adhesion amount is at least about 150% of a control. In some embodiments the enhanced adhesion amount is at least about 300% of a control. In some embodiments the enhanced adhesion amount is at least about 1000% of a control. In some embodiments the enhanced adhesion amount is at least about 1500% of a control. In some embodiments the enhanced adhesion amount is at least about 2000% of a control. In some embodiments the enhanced adhesion amount is at least about 2700% of a control. In some embodiments the enhanced adhesion amount is at least about 4700% of a control. In various embodiments, the effective amount of integrin-stimulating peptide bound to the implant is the amount sufficient to achieve any of the foregoing enhanced adhesion amounts, or amounts within ranges of the foregoing, such as 150-300% of a control, 1000-1500% of a control, etc.

In some embodiments, the biomaterial may have a ceramic material such as a zeolite incorporated in the resin and/or on the surface of the resin. The presence of the zeolite imparts hydrophilicity and a negative charge to the device, and provides available ion-exchange sites for the optional incorporation of metal ions that can be eluted into the host after implantation. Thus, the zeolite optionally may be loaded with one or more therapeutic metal ions that exhibit therapeutic properties when implanted into a body and exposed to bodily fluid or tissue. Suitable ions include silver, copper, zinc, mercury, tin, magnesium, lead, gold, bismuth, cadmium, chromium, strontium and thallium ions, calcium, silicon or combinations of one or more of the foregoing. Such devices, when implanted into a body and exposed to bodily fluid, may elute metal ions in a therapeutically or prophylactically effective amount. In certain embodiments, the source of therapeutic or prophylactic activity includes ion-exchangeable cations contained in a zeolite. The metal ions may include one or more divalent cations that contribute to integrin-ligand binding affinity.

Zeolites can be obtained in master batches of pellets of resin, e.g., low density polyethylene, polypropylene, ultra-high molecular weight polyethylene or polystyrene, containing suitable amounts of zeolite particles, usually 20 wt. % of zeolite particles. When provided in this form, the pellets of resin containing the zeolite particles can be easily mixed with resins used to make the implants or used to make coatings to be applied to the implants, as set forth in U.S. Pat. No. 6,582,715, the disclosure of which is hereby incorporated by reference. Typical amounts of zeolite particles incorporated in an implant resin range from 0.01 to 20 wt. %, more preferably 0.01 to 10 wt. %, even more preferably 0.01 to 8.0 wt. %, most preferably 0.1 to 5.0 wt. %. The method used to coat or otherwise incorporate the ceramic into the resin is not particularly limited, and can include spraying, painting or dipping. When compounded into PEEK, for example, the PEEK should be protected from sources of moisture and contamination. The compounding can be carried out by blending. The ceramic species can be a surface coating, can be incorporated or embedded into the thermoplastic resin, or can be both a surface coating and incorporated or embedded into the resin. Similar amounts may be incorporated into or infused into the suture ring fabric or textile, where present.

In certain embodiments, disclosed are methods of imparting therapeutic activity to devices by functionalizing the surface of the device including immobilizing one or more peptides on the surface, and optionally also by controlling the delivery of certain cations through ion-exchange via a ceramic material such as zeolite incorporated in the device. The presence of the ceramic material such as zeolite at the exposed surface of the device proactively modulates the immune-mediated host tissue reaction to the presence of the implant. The presence of the ceramic material such a zeolite reduces the immune response of the host to the implant by promoting pro-regenerative immune cell phenotypes that support constructive tissue remodeling, e.g., causing a reduction of deleterious release of cytokines, such as interleukin 2, etc., upon implantation. A rapid transition in the host from M1 proinflammatory macrophage phenotype to the M2 macrophage phenotype occurs, thereby minimizing fibrous encapsulation and reducing pain. The term “exposed surface” is intended to include one or more surfaces of an implantable device that when implanted into the body of a host, is exposed to or in contact with body tissue and/or bodily fluids of the host.

The hydrophilicity imparted by the ceramic material such as zeolite results in an engineered biomaterial that interacts with the tissue of the patient and induces fusion. The presence of the ceramic material such as zeolite also results in a rapid transition from M1 proinflammatory macrophage phenotype to the M2 macrophage phenotype, thereby minimizing fibrous encapsulation and facilitating the deposition of cite appropriate tissue ultimately yielding constructive and functional tissue remodeling.

In certain embodiments, the thermoplastic polymer is PEEK.

The configuration of the mechanical heart valve is not particularly limited, but typically includes a valve body or support having a central passageway, a fixation member such as an outer sewing ring or annulus extending from the valve body and configured to permit the valve to be sutured into operative position in the region where the original valve of the host was removed, and a valve mechanism, typically a trileaflet or bileaflet valve mechanism, such as in the St. Jude's valve and the Regent™ and Master Series heart valves commercially available from Abbott. The valve mechanism is operably configured to allow blood flow through the passageway in a first position, and block blood flow through the passageway in a second position. The fixation member is conventionally composed of or covered with a biocompatible fabric suitable to be attached to a heart annulus with sutures. The valve mechanism may be composed of any of the aforementioned materials, alone or in combination.

In some embodiments, the fixation member is a sewing ring and is an annular ring that extends radially outwardly from the valve body. In some embodiments, the fixation member is a sewing ring and is composed of fabric, and the fabric is infused with zeolite. Suitable fabrics include synthetic fibers such as woven fabrics, non-woven fabrics, cloth, Dacron® fabric, PTFE fibers, ePTFE, nylon, polyester, polypropylene, etc. Infusion techniques are known in the art. For example, fibers may be extruded from a hot melt that contains zeolite, such as at concentrations of from about 5 to about 20%, preferably about 10 to about 15%, most preferably about 12%. The melt is extruded and the extruded fibers are cooled. In the process of extrusion and cooling the fiber optionally may be run through a solution of metal nitrate (e.g., silver nitrate) at a pH of about 6. The sodium ions in the exposed zeolite will exchange with silver from the silver nitrate solution depending on the equilibrium, relative concentrations and exposure times. The loading extent of the metal into the zeolite carrier can be determined by immersing a I g sample of the fiber in 50 ml of 0.8% sodium nitrate solution and determining the elution extent by ICP OES, as is known in the art.

In certain embodiments the biomaterial may optionally be formulated by blending a base polymer, such as PEEK, with a negatively charged zeolite. The zeolite changes the surface topography, charging characteristics, and pH of the resulting composite in a predictable, suitable manner for the surgical environment and long-term healing of the host patient into which the device is implanted. Attributes imparted by the zeolite include biocompatibility, negative charge, hydrophilicity, preferential polarization towards an M2 phenotype post-implantation, and promotion of cell adhesion. Attributes that may be provided by the base polymer such as PEEK include radiolucency, biocompatibility, durability and versatility. The resulting composite blend provides a uniform material construct and excellent workability.

In embodiments where zeolite is present, whether in the valve body, the sewing ring fabric, or both, particularly compelling is the ability of the zeolite to reduce or eliminate the immune response that is generated when a naked polymer is implanted in a host. It is a well-recognized problem that the human immune system reacts to the presence of the naked polymer as a foreign, unnatural substance, and as a damage/danger associated molecular pattern (DAMP). Consequently, the human body responds to the presence of naked PEEK (and other synthetic polymer resins) by encapsulating it, causing bone resorption, and initiating a pain response. This is believed to be directly related to the hydrophobic, uncharged and water repellant nature of the naked resin. Adding zeolite to the polymer and/or sewing ring fabric increases proliferation, differentiation and transforms growth factor beta production in normal adult human osteoblast-like cells. The hydrophilic surface of the resulting implant down-regulates pro-inflammatory cytokines interleukin 1 & 6, which modulates the immune response, facilitates wound healing, allows for early cell adhesion, and reduces pain. IL1-Beta upregulates inflammatory immune-response, and IL6-Beta has been shown to have a direct relation to pain.

Composites of zeolite with PEEK and other suitable resins produce a more hydrophilic and negatively charged surface which is less favorable to bacterial adhesion. The presence of the zeolite results in a rapid transition (e.g., faster than the transition that occurs in the absence of zeolite) from M1 proinflammatory macrophage phenotype to the M2 macrophage phenotype, thereby minimizing fibrous encapsulation, and hence likely less bacterial seeding of the valve associated with bacteremia/sepsis.

In certain embodiments, ceramic particles may be incorporated into the polymer interbody cage to form a composite polymer resin/zeolite blend. In some embodiments, the implant includes PEEK resin, and ceramic particles such as zeolite are uniformly incorporated into the main body region of the resin.

In some embodiments, either natural zeolites or synthetic zeolites may be used to make the zeolites used in the embodiments disclosed herein. “Zeolite” is an aluminosilicate having a three-dimensional skeletal structure that is represented by the formula: XM_{2/n}O·Al₂O₃·YsiO₂·ZH₂O, wherein M represents an ion-exchangeable ion, generally a monovalent or divalent metal ion, n represents the atomic valency of the (metal) ion, X and Y represent coefficients of metal oxide and silica respectively, and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. A-type zeolites are particularly preferred, such as 4A zeolite having particle size ranges from 1 to 10 microns with a narrow distribution of about 4 microns.

Other ceramics and metal glasses are also envisaged instead of zeolite and are within the scope of the embodiments disclosed herein. For example, zirconium phosphate, bioglass (e.g., bioactive glass particulate material or fibers, such as 45S5 (calcium sodium phosphosilicate), 58S and S70C30 bioactive glass) or silver glass could be used.

In certain embodiments where the main body region of the article also is to include zeolite, for example, fine zeolite powder may be incorporated into a powder of the thermoplastic polymer. For example, 4 micron powder of a 4A zeolite may be incorporated into molten PEEK powder that has a particle diameter of between about 10 to about 100 microns. In some embodiments, the incorporation of the zeolite into the polymer is carried out by thorough mixing the dry components at room temperature until the resulting composition is uniform by visual inspection. In some embodiments a drum roller can be used to carry out the mixing process.

The powder formulation may include the polymer, such as PEEK, and zeolite, or metal-loaded zeolite, such as silver and/or copper zeolite. Other bioactive agents also may be included.

In certain embodiments, when metal cation is used, the metal cation is present at a level below the ion-exchange capacity in at least a portion of the zeolite particles. In some embodiments, the amount of zeolite mixed with the polymer may range from about 5 to 50 wt. %, more preferably about 10 to 20 wt. %. The amount of metal ions, if present, in the zeolite should be sufficient such that they are present in a therapeutically effective amount when implanted into the body of a patient. For example, suitable amounts can range from about 0.1 to about 20 or 30% of the exposed zeolite (w/w %). These levels can be determined by complete extraction and determination of metal: in the extraction solution by atomic absorption or ICP OES. Preferably the ion-exchanged metal cations, if present, are present at a level less than the ion-exchange capacity of the ceramic particles. The amount of ammonium ions is preferably limited to from about 0.5 to about 15 wt. %, more preferably 1.5 to 5 wt. %. For applications where strength is not of the utmost importance the loading of zeolite can be taken as high as 50%, whether metal ion is incorporated or not. At such loadings the permeation of metal ions, where present, can permeate well below the surface layer due to interparticle contact, and much greater loadings of metal ions are possible.

In some embodiments, the ceramic material such as zeolite can be post-loaded with metal ions after it has been incorporated into the resin or fabric or both. Metal ion salt solutions, such as nitrates, acetates, benzoates, carbonates, oxides, etc., can be used to accomplish this. Addition of nitric acid to the infusion solution also may be advantageous in that it can etch the surface of the implant, providing additional surface area for ion exchange. That is, the zeolite may be charged with metal ions at a temperature between about 0 and 100° C., preferably about room temperature) from a metal ion source such as an aqueous metal ion solution, such as silver nitrate, copper nitrate and zinc nitrate, alone or in combination. Cooling to lower temperatures gives lower loading rates but higher stability. Loading at even higher temperatures can be carried out at a faster rate by maintaining the system under pressure, such as in a pressure cooker or autoclave. The content of the ions can be controlled by adjusting the concentration of each ion species (or salt) in the solution.

For example, zeolite can be loaded with metal ions by bringing the composite material into contact with an aqueous mixed solution containing ammonium ions and antimicrobial and/or therapeutic metal ions such as silver, copper, zinc, strontium, etc. These materials will strongly inhibit attachment of microorganisms and can accelerate healing and reduce inflammation and likelihood of permanent bacterial seeding of the valve. By loading metal ions at these temperatures, deleterious oxidation of the metal ions that occurs at higher processing temperatures is reduced or eliminated. The most suitable temperatures at which the infusion can be carried out range from 5° C. to 75° C., but higher temperatures may also be used even above 100° C. if the reaction vessel is held under pressure. Higher temperatures will show increased infusion rates but lower temperatures may eventually produce more uniform and higher loadings. The pH of the infusion solution can range from about 2 to about 11 but is preferably from about 4 to about 7. Suitable sources of ammonium ions include ammonium nitrate, ammonium sulfate and ammonium acetate. Suitable sources of the metal ions include: a silver ion source such as silver nitrate, silver sulfate, silver perchlorate, silver acetate, diamine silver nitrate and diamine silver nitrate; a copper ion source such as copper(II) nitrate, copper sulfate, copper perchlorate, copper acetate, tetracyan copper potassium; a zinc ion source such as zinc(II) nitrate, zinc sulfate, zinc perchlorate, zinc acetate and zinc thiocyanate.

Preferably, the implant includes PEEK resin, and ceramic particles optionally are incorporated into the resin such that a negative charge is imparted to an exposed surface of the resin. The term “exposed surface” is intended to include one or more surfaces of an implantable device that when implanted, is exposed to or in contact with body tissue and/or fluids.

In certain embodiments, the implant comprises a polymer resin that has been functionalized with a therapeutically effective amount of one or more peptides or peptidomimetics (any oligomeric sequence designed to mimic a peptide structure and/or function but whose backbone is not solely based on alpha-amino acids). Preferably the peptide is an integrin-stimulating peptide. Preferably the peptide includes the RGD sequence of amino acids. Most preferably the peptide is linear or cyclic RGD (CRGD). Suitable peptides include RGD, RGDS, GRGD, GRGDS, GRGDSPC, GRGFSPC and cyclo-RGDFV. The RGD motif may be flanked by other amino acids as well. In certain embodiments, the peptide interacts with cells adjacent or in proximity to the exposed surface of the implant when the implant is implanted in a host. In some embodiments, the interaction promotes the adhesion of cells to the implant.

In various embodiments, the implant is subjected to surface treatment, such as to enhance the attachment or immobilization of the peptide. Preferably the surface treatment is a plasma treatment which raises the surface energy. Plasma surface treatment can assist in creating chemically active functional groups, such as amine, carbonyl, and carboxyl hydroxyl groups, to improve interfacial adhesion. Most preferably the surface treatment is corona discharge treatment in order to enhance the binding or immobilization of the peptide sequence to or on the implant surface, particularly the exposed surface. Corona discharge is an electrical phenomenon where a gas surrounding a high voltage electrode forms an ionized gaseous plasma; application of high voltage to a conductor can lead to the ionization of a surrounding medium due to the high value of the electric field around it. Corona discharge is a specific sub-type of plasma generated by the ionization of air molecules at atmospheric pressure. Corona is a stream of charged particles such as electrons and ions that is accelerated by an electric field. It is generated when a space gap filled with air or other gases is subjected to a sufficiently high voltage to set up a chain reaction of high-velocity particle collisions with neutral molecules resulting in the generation of more ions.

In various embodiments, the implant article is exposed to a corona discharge produced by high-frequency, high-voltage alternating current. Exposing the implant surface to corona discharge enhances the ability to functionalize the surface (e.g., by attachment of integrin-stimulating peptides), which in turn enhances cell adhesion and surgical success. In some embodiments of the corona discharging treatment, the implant article may be introduced into an air gap between two electrodes, one of which is energized with a high voltage electrical field and the other of which is grounded. High voltage power is applied, the air in the gap becomes ionized from the acceleration of electrons to form a gaseous conductor comprising corona. The ionized air gap induces an electron avalanche which in turn creates oxidative molecules such as ozone. The ozone oxidizes the surface of the implant article and increases its surface energy.

One suitable corona discharge treatment apparatus is a 25KVA DC Charge machine commercially available from Tantec. Other voltages and currents which produce similar corona effects to activate the implant surface also may be used, including apparatus capable of AC corona discharge treatment.

Preferably the surface treatment is applied to the entire surface of the implant. Treatment times are not particularly limited, with suitable treatment times ranging between about 15 seconds to about 1 minute, with 30 seconds being particular suitable. Corona activated surfaces have a tendency to revert to their original state with time, so preferably subsequent surface functionalization such as with an RGD peptide is carried out as soon as possible (e.g., within 1 to 5 minutes) after the surface is activated by the corona discharge treatment. However, adsorption of the peptide is likely to be effective even hours after the surface is activated.

Once the implant has been plasma treated, the implant may be contacted with an activating agent. Activating agents can provide the attachment of functional groups by reacting with the energized implant material surface, and can immobilize the integrin-stimulating peptide by providing covalent linkage between the polymer (e.g., PEEK) and the integrin-stimulating peptide (e.g., peptides with the motif RGD). These functional groups may enhance cell adhesion on their own in addition to providing even stronger bonding sites for integrin-stimulating peptides. These activating agents can include those with functional groups such as amino groups, hydroxyl groups and/or carboxyl groups, such as amino acids, amines, amides and other reactive bases. Acids can also provide an enhancement of the reactivity of the polymer by reacting with the surface and producing bonding functionality. Suitable activating agents include ammonium hydroxide, citric acid, nitric acid and sulfuric acid. Acids can be used at high concentration for a short period of time or at a lower concentration, 1 to 5% for a protracted period of time. Heating will increase the reaction rate. At a longer time exposure, from 5 to 25% sulfuric acid is likely to be effective particularly if the temperature is raised above room temperature. The activating agents can provide a stable covalent linkage between the integrin-stimulating peptide and the polymer resin.

If the activators are present during the discharge process, it is likely that their reactivity with the polymer will be strongly enhanced. For example, if sulfuric acid solution is applied during the discharge process, its reactivity is likely to be much stronger and likely to be effective at a low concentration, such as 2%. Activating agents such as ammonia gas or sulfur dioxide can be used in their gaseous form such as when the corona discharge treatment is carried out in a closed chamber.

Corona discharge or plasma treatment of a plastic provides open reactive functional groups and open bonds to which the peptides and/or activating agent can bond, just as the treatment which is used on plastic film to enhance printability and a substrate to which the printing inks can bond strongly.

In various embodiments, after being exposed to an activating agent, the implant is contacted with or otherwise exposed to a peptide solution to bind the peptide to the implant. Once the peptide is exposed to the activated polymer composite surface it is expected to react instantaneously with the reactive species on the surface of the composite. However, longer time exposure is not expected to compromise the effectiveness of the attachment of the peptide to the surface. The bonds once formed can withstand sterilization and washing with water or alcohol. One suitable solvent for delivery of the peptide to the material surface is distilled water at ambient temperature.

In some embodiments, the implant material may be subjected to surface treatment (e.g., plasma or corona discharge treatment) and functionalized such as by exposure to peptide in an automated process. For example, the one or more implant devices may be passed through a 360 degree corona discharge treatment, such as with a SyrinTEC corona treatment process commercially available from Tantec. An activating solution such as ammonium hydroxide may be then applied to the implant, such as by spraying the implant with or dipping the implant in the activating solution. Excess solution may be removed, such as by shaking or blowing, and then the peptide may be applied to the implant such as by spraying or dipping the implant into a solution of the peptide. The implant may be then rinsed, preferably multiple times (e.g., three times) to remove unbonded residual peptide. It may be then dried, sanitized (e.g., irradiated or autoclaved) and sterile packed.

The resulting device may be introduced into the body surgically. Suitable hosts include mammals, including humans, canines, felines, livestock, primates, etc.

The rate of release of therapeutic metal ions, if present, is governed by the extent of loading of the polymer with ceramic such as zeolite and the extent to which the exposed zeolite is charged with metal ions. The electrolyte concentration in host blood and body fluids is relatively constant and will cause ion exchange with ions such as silver, copper and zinc, etc. from the surface of the implant, which deactivate or kill gram positive and gram negative organisms, including E. coli and Staphylococcus aureus. Effective antibacterial control (e.g., a six log reduction of microorganisms) is achieved even at low metal ion concentrations of 40 ppb. Divalent cations can be used to enhance integrin activation.

Surface occupancy of the ceramic such as zeolite can be determined indirectly by post loading the ceramic with a therapeutic metal ion, removing non absorbed metal by thorough rinsing and determining the amount which can be extracted into a 1% sodium nitrate solution by ICP OES. Comparison elution from a composite without the enhanced zeolite addition to the exposed surface region will give an indication of the extent of the surface enhancement of zeolite concentration.

In some embodiments, the implant can be engineered such that a portion of the exposed surface less than the whole includes zeolite, with the remainder being naked resin (e.g., naked PEEK) devoid of zeolite. All or part of the portion of the exposed surface devoid of zeolite can then be functionalized as discussed above, e.g., surface activated, optionally addition of an activating agent, and optionally addition of an integrin-stimulating peptide such as RGD. In this way, the advantages of both the zeolite and surface functionalization and/or activation are realized.

### EXAMPLE 1

### Specific Aim

To test the individual and combinatorial effect of RGD peptides, corona discharge surface treatment and the effect of acid and base treatment (sulfuric, nitric, phosphoric and citric as acids and ammonia as a base) in enhancing cell attachment and response on PEEK surfaces relative to a native PEEK surface which was untreated.

### Sample Preparation

Coupons of naked PEEK (commercially supplied Solvay PEEK rod that has been extruded from pellets into a one meter long rod 30 mm diameter) were used. Eleven discs were cut from the rod with a band saw to a thickness of about 2 mm to form sample coupons and were treated with the regimens described below. All samples were sterilized using propanol and oven dried at 120° F. to make sure that no trace propanol was left behind on the samples.

Sample 1 was an untreated PEEK coupon.

Sample 2 was a PEEK coupon coated with a one ml aliquot of RGD peptide solution on the upper surface. The RGD peptide was a dry white powder obtained from Abcam of Cambridge, Massachusetts, and was diluted with distilled water to produce a 10 millimolar solution. The coated sample coupon was allowed to sit in a petri dish overnight and the rinsed three times with distilled water to remove any residual unbonded peptide. Unbonded peptide has the potential to reduce cell adhesion since it can bond to the active site on the integrin and prevent it from bonding to the surface.

Sample 3 was a PEEK coupon subjected to a corona discharge treatment for 30 seconds under the following conditions. The power supply used to produce the corona discharge was from an Eastwood's powder coating unit, “DUAL-VOLTAGE POWDER COATING SYSTEM WITH HIGH-FREQUENCY PULSE TECHNOLOGY”, Part No. 11676, Complete Dual-Voltage, Pulse Technology Power Supply (requires grounded 120 VAC/60-50 hz.) unit with:
6 ft. 120 volt, 15 amp electrical input cord.
6 ft. lead with ground clamp which is attached to the part to be powder coated.
Remote activation switch with 6 ft. lead (applies voltage to emitter: hold-on, release-off)
8 ft. high voltage gun power lead (converts 110-120 VAC to 15 KVDC or 25 KVDC (no user-serviceable parts))

An insulated wire lead with two crocodile clips was attached to the emitter and the second end of the lead was attached to the sample coupon. A second lead was attached to the earth lead with the second end of the lead being used to complete the circuit by bringing it close to or touching it to the sample specimen. The discharge was allowed to continue for 30 seconds as the lead was manually moved over and around the surface of the disk to ensure that all of the sample was exposed to the discharge. The power supply was set at the 25 KVDC setting.

The surface of the PEEK coupon was wetted with distilled water and again exposed to a corona discharge for 30 seconds under the same conditions as above. The sample coupon was shaken dry and its upper surface was coated with a 1 ml aliquot of the same RGD peptide solution as above. The sample coupon was allowed to sit in a petri dish overnight and was then rinsed three times with distilled water to remove any residual unbonded peptide.

Sample 4 was a PEEK coupon exposed to a dry corona discharge using the same parameters for Sample 3 for 30 seconds. It was then thoroughly wetted with a 2% ammonium hydroxide solution and again exposed to a corona discharge for 30 seconds, again using the same parameters as above. The sample coupon was shaken dry and its upper surface coated with a 1 ml aliquot of the same RGD peptide solution as above. The sample coupon was allowed to sit in a petri dish overnight and rinsed three times with distilled water to remove any residual unbonded peptide.

Sample 5 was a PEEK coupon exposed to a dry corona discharge using the same parameters for Sample 3 for 30 seconds. It was then thoroughly wetted with a 1% solution of citric acid and again exposed to a corona discharge for 30 seconds, again using the same parameters as above. The sample coupon was shaken dry and then its upper surface was coated with a 1 ml aliquot of the same RGD peptide solution as above. The sample coupon was allowed to sit in a petri dish overnight and then rinsed three times with distilled water to remove any residual unbonded peptide.

Sample 6 was a PEEK coupon shaken in a sealed container of fuming nitric acid for 5 minutes. The sample coupon was then rinsed free of acid and then rinsed again three times with distilled water to remove all residual nitric acid and dried. The sample coupon was allowed to sit in a petri dish overnight. It was then rinsed three times with distilled water to keep the handling consistent with the other samples.

Sample 7 was a PEEK coupon was shaken in a sealed container of fuming nitric acid for 5 minutes. The coupon was then rinsed free of acid and then rinsed again three times with distilled water to remove all residual nitric acid and dried. The sample coupon was exposed to a dry corona discharge using the same parameters for Sample 3 for 30 seconds, and then thoroughly wetted with distilled water and again subjected to a corona discharge for 30 seconds, again using the same parameters as above. The water was shaken from the surface and a 1 ml aliquot of the same RGD peptide solution as above was carefully applied to the upper surface of the coupon which had been placed in a petri dish. The sample was allowed to sit in a petri dish overnight. It was then rinsed three times with distilled water to remove any residual unbonded peptide.

Sample 8 was a PEEK coupon that was shaken in a sealed container of concentrated (98%) sulfuric acid for 5 minutes. The sample coupon was then rinsed free of acid and then again rinsed three times with distilled water to remove all residual acid. The water was shaken from the surface and a 1 ml aliquot of the same RGD peptide solution as above was carefully applied to the upper surface of the coupon which had been placed in a petri dish. The sample was allowed to sit in a petri dish overnight. It was then rinsed three times with distilled water to maintain consistency with the treatment of other samples.

Sample 9 was PEEK coupon that was s shaken in a sealed container of concentrated (98%) sulfuric acid for 5 minutes. The coupon was then rinsed free of acid and then again rinsed there times with distilled water to remove all residual acid and dried. The coupon was exposed to a dry corona discharge using the same parameters for Sample 3 for 30 seconds, and then thoroughly wetted with distilled water and again subjected to a corona discharge for 30 seconds, again using the same parameters as above. The water was shaken from the surface and a 1 ml aliquot of same RGD peptide solution as above was carefully applied to the upper surface of the coupon which had been placed in a petri dish. The sample was allowed to sit in a petri dish overnight. It was then rinsed three times with distilled water to remove any residual unbonded peptide.

Sample 10 was a PEEK coupon that was shaken in a sealed container of 1 molar phosphoric acid for 5 minutes. The coupon was then rinsed free of acid and then again rinsed three times with distilled water to remove all residual acid and dried. The sample was allowed to sit in a petri dish overnight. It was then rinsed three times with distilled water to maintain consistency with the treatment of other samples.

Sample 11 was a PEEK coupon that was shaken in a sealed container of concentrated I molar phosphoric acid for 5 minutes. The coupon was then rinsed free of acid and then again rinsed three times with distilled water to remove all residual acid and dried. The coupon was exposed to a dry corona discharge using the same parameters for Sample 3 for 30 seconds, thoroughly wetted with distilled water and again subjected to a corona discharge for 30 seconds, again using the same parameters as set forth above. The water was shaken from the surface and a 1 ml aliquot of RGD peptide solution as above was carefully applied to the upper surface of the coupon which had been placed in a petri dish. The sample was allowed to sit in a petri dish overnight. It was then rinsed three time with distilled water to remove any residual unbonded peptide.

Each of samples 1 through 11 were retained for about a week and then rinsed with 90% 2-propanol to sterilize, followed by drying in an oven at 120° Centigrade for 30 minutes to ensure that no trace propanol remained prior to inoculation with cells. The geometrical surface area of the sample coupons was 7.065 cm².

### Cell Proliferation

An alamarBlue® assay was used to quantitatively measure cell proliferation. Saos-2 cells (ATCC HTB-85) grown in McCoy's 5A Media (ATCC 30-2007) supplemented with 15% fetal bovine serum (FBS), were seeded at a density of 1.35*10⁵ onto PEEK (control) and the various treatment arms and positive control as enlisted above. All of the samples were bathed in 4 mls of media. On day 3, all the implants and positive control were removed from their wells and incubated in fresh 6 well plates and were trypsinized to detach the cells that were attached to the implants. After detachment, the cells in trypsin were centrifuged at 300 g for 5 mins and were resuspended in 2 ml of fresh cell media after removal of the trypsin.

An alamarBlue® assay was performed by adding 200 ul of Alamar blue dye to the samples (10% of cell media volume, 1:10 ratio of dye: media) and fluorescence was allowed to develop for 24 hours.

The alamarBlue® assay is designed to measure quantitatively the proliferation of various human cell lines, bacteria and fungi. The alamarBlue® assay incorporates a fluorometric/colorimetric growth indicator based on detection of metabolic activity. After 24 hours, the fluorescence of the individual samples and controls were measured at 590 nm (emission) after a 560 mm (excitation). Unknown samples were converted to cell numbers using a known cell number standard curve using a generated quadratic regression equation as shown in FIG. 1.

### Results

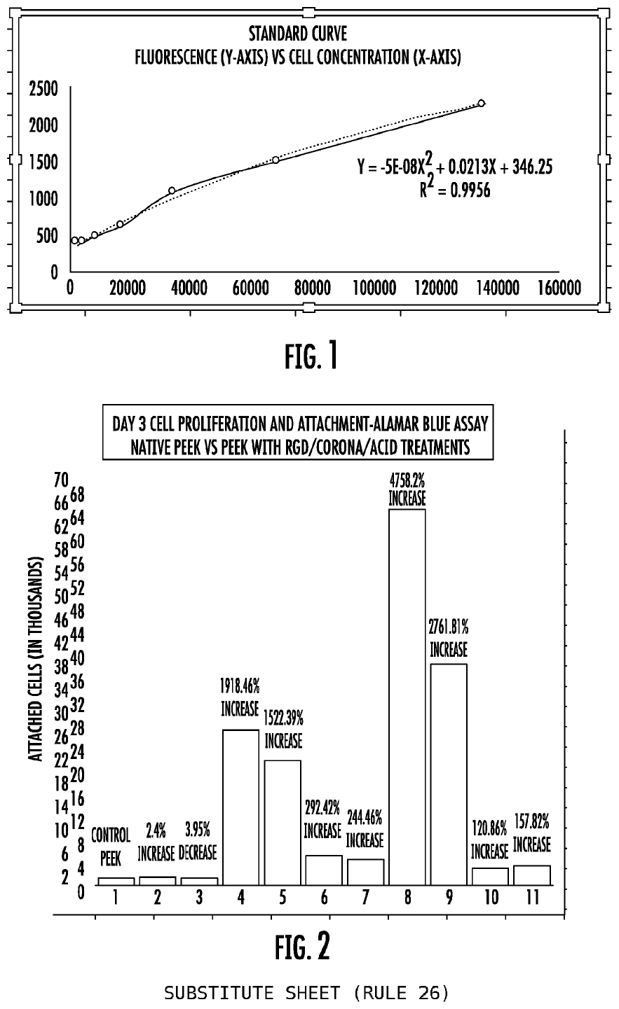

It is clear from the results as shown in FIG. 2 (% increase or decrease in cellular attachment is shown in the graph) that the treatments two and three were not effective in enhancing surface attachment of cells relative to the control and showed either an insignificant increase or decrease in cellular attachment.

The most effective treatments were samples 4, 5, 8 and 9, which showed an exaggerated effect in increasing the cell response. Sample 4 demonstrated a 1918% increase; Sample 5 demonstrated a 1522% increase; Sample 8 demonstrated a 4578% increase; and Sample 9 exhibited a 2761% increase in cell coverage over the control. All the remaining samples exhibited a statistically significant increase in cell attachment relative to the control. This demonstrates substantial cause and effect in showcasing the efficacy of such treatments in enhancing the cell attachment characteristics to PEEK.

In summary, the effects of 1) ammonia treatment, corona discharge and RGD peptide attachment onto PEEK (Sample 4); 2) citric acid treatment, corona discharge and RGD peptide attachment onto PEEK (Sample 5); 3) Treatment with sulfuric acid and RGD peptide (Sample 8) and 4) sulfuric acid treatment, corona discharge and RGD peptide attachment (Sample 9) were all exaggerated in enhancing cell response on these treated PEEK surfaces relative to native PEEK.

### EXAMPLE 2

### Specific Aim

To test the individual and combinatorial effect of RGD peptides, corona discharge surface treatment and the effect of acid and base treatment (sulfuric, nitric, phosphoric and citric as acids and ammonia as a base) in enhancing cellular attachment and response on PEEK-zeolite composite surfaces relative to a native PEEK-zeolite surface which was untreated.

### Methods

A composite blend of PEEK and zeolite (12% 4A zeolite) (“ZFUZE” coupon) that had been extruded from compounded pellets into a one meter 30 mm diameter rod was used. Eleven discs were cut from the rod with a band saw to a thickness of about 2 mm to form sample coupons and were treated with the regimens described below. All samples were sterilized using propanol and oven dried at 120° F. to make sure that no trace propanol was left behind on the samples.

Sample 1: PEEK-zeolite composite untreated coupon (native “ZFUZE” coupon) (control)*

Sample 2: A PEEK zeolite composite coupon coated with a one ml aliquot of RGD peptide solution on the upper surface. The RGD peptide was a dry white powder obtained from Abcam of Cambridge, Massachusetts, and was diluted with distilled water to produce a 10 millimolar solution. The coated sample coupon was allowed to sit in a petri dish overnight and the rinsed three times with distilled water to remove any residual unbonded peptide. Unbonded peptide has the potential to reduce cellular adhesion since it can bond to the active site on the integrin and prevent it from bonding to the surface.

Sample 3: A PEEK zeolite composite coupon was treated with coronal discharge while dry and then wetted with distilled water and treated again with corona discharge, followed by rapidly coated with RGD peptide solution under the following conditions. The power supply used to produce the corona discharge was from an Eastwood's powder coating unit, “DUAL-VOLTAGE POWDER COATING SYSTEM WITH HIGH-FREQUENCY PULSE TECHNOLOGY”, Part No. 11676, Complete Dual-Voltage, Pulse Technology Power Supply (requires grounded 120 VAC/60-50 hz.) unit with:
6 ft. 120 volt, 15 amp electrical input cord.
6 ft. lead with ground clamp which is attached to the part to be powder coated.
Remote activation switch with 6 ft. lead (applies voltage to emitter: hold-on, release-off).
8 ft. high voltage gun power lead (converts 110-120VAC to 15 KVDC or 25 KVDC (no user-serviceable parts))
   An insulated wire lead with two crocodile clips was attached to the emitter and the second end of the lead was attached to the sample coupon. A second lead was attached to the earth lead with the second end of the lead being used to complete the circuit by bringing it close to or touching it to the sample specimen. The discharge was allowed to continue for 30 seconds as the lead was manually moved over and around the surface of the disk to ensure that all of the sample was exposed to the discharge. The power supply was set at the 25 KVDC setting.

The surface of the sample coupon was wetted with distilled water and again exposed to a corona discharge for 30 seconds under the same conditions as above. The sample coupon was shaken dry and its upper surface was coated with a 1 ml aliquot of the same RGD peptide solution as above. The sample coupon was allowed to sit in a petri dish overnight and was then rinsed three times with distilled water to remove any residual unbonded peptide.

Sample 4: A PEEK zeolite composite coupon treated with corona discharge in ammonia solution then treated with RGD peptide solution. The sample coupon was exposed to a dry corona discharge using the same parameters for Sample 3 for 30 seconds. It was then thoroughly wetted with a 2% ammonium hydroxide solution and again exposed to a corona discharge for 30 seconds, again using the same parameters as above. The sample coupon was shaken dry and its upper surface coated with a 1 ml aliquot of the same RGD peptide solution as above. The sample coupon was allowed to sit in a petri dish overnight and rinsed three times with distilled water to remove any residual unbonded peptide.

Sample 5: A PEEK zeolite composite coupon treated with corona discharge in citric acid solution then treated with RGD peptide solution. The sample coupon was exposed to a dry corona discharge using the same parameters for Sample 3 for 30 seconds. It was then thoroughly wetted with a 1% solution of citric acid and again exposed to a corona discharge for 30 seconds, again using the same parameters as above. The sample coupon was shaken dry and then its upper surface was coated with a 1 ml aliquot of the same RGD peptide solution as above. The sample coupon was allowed to sit in a petri dish overnight and then rinsed three times with distilled water to remove any residual unbonded peptide.

Sample 6: A PEEK zeolite composite coupon treated with nitric acid. The sample coupon was shaken in a sealed container of fuming nitric acid for 5 minutes. The sample coupon was then rinsed free of acid and then rinsed again three times with distilled water to remove all residual nitric acid and dried. The sample coupon was allowed to sit in a petri dish overnight. It was then rinsed three times with distilled water to keep the handling consistent with the other samples.

Sample 7: A PEEK zeolite composite coupon treated with nitric acid, rinsed, corona discharge treated and then treated with RGD peptide solution. The sample coupon was shaken in a sealed container of fuming nitric acid for 5 minutes. The coupon was then rinsed free of acid and then rinsed again three times with distilled water to remove all residual nitric acid and dried. The sample coupon was exposed to a dry corona discharge using the same parameters for Sample 3 for 30 seconds, and then thoroughly wetted with distilled water and again subjected to a corona discharge for 30 seconds, again using the same parameters as above. The water was shaken from the surface and a 1 ml aliquot of the same RGD peptide solution as above was carefully applied to the upper surface of the coupon which had been placed in a petri dish. The sample was allowed to sit in a petri dish overnight. It was then rinsed three times with distilled water to remove any residual unbonded peptide.

Sample 8: A PEEK zeolite composite coupon treated with sulfuric acid and RGD peptide solution. The sample coupon was shaken in a sealed container of concentrated (98%) sulfuric acid for 5 minutes. The sample coupon was then rinsed free of acid and then again rinsed three times with distilled water to remove all residual acid. The water was shaken from the surface and a 1 ml aliquot of the same RGD peptide solution as above was carefully applied to the upper surface of the coupon which had been placed in a petri dish. The sample was allowed to sit in a petri dish overnight. It was then rinsed three times with distilled water to maintain consistency with the treatment of other samples.

Sample 9: A PEEK zeolite composite coupon treated with sulfuric acid, rinsed, treated with corona discharge and then treated with RGD peptide solution. The sample coupon was shaken in a sealed container of concentrated (98%) sulfuric acid for 5 minutes. The coupon was then rinsed free of acid and then again rinsed there times with distilled water to remove all residual acid and dried. The coupon was exposed to a dry corona discharge using the same parameters for Sample 3 for 30 seconds, and then thoroughly wetted with distilled water and again subjected to a corona discharge for 30 seconds, again using the same parameters as above. The water was shaken from the surface and a 1 ml aliquot of same RGD peptide solution as above was carefully applied to the upper surface of the coupon which had been placed in a petri dish. The sample was allowed to sit in a petri dish overnight. It was then rinsed three times with distilled water to remove any residual unbonded peptide.

Sample 10: A PEEK zeolite composite coupon treated with phosphoric acid. The sample coupon was shaken in a sealed container of 1 molar phosphoric acid for 5 minutes. The coupon was then rinsed free of acid and then again rinsed three times with distilled water to remove all residual acid and dried. The sample was allowed to sit in a petri dish overnight. It was then rinsed three times with distilled water to maintain consistency with the treatment of other samples.

Sample 11: A PEEK zeolite composite coupon treated with phosphoric acid, rinsed, treated with corona discharge and then treated with RGD peptide solution. The sample coupon was shaken in a sealed container of concentrated I molar phosphoric acid for 5 minutes. The coupon was then rinsed free of acid and then again rinsed three times with distilled water to remove all residual acid and dried. The coupon was exposed to a dry corona discharge using the same parameters for Sample 3 for 30 seconds, thoroughly wetted with distilled water and again subjected to a corona discharge for 30 seconds, again using the same parameters as set forth above. The water was shaken from the surface and a 1 ml aliquot of RGD peptide solution as above was carefully applied to the upper surface of the coupon which had been placed in a petri dish. The sample was allowed to sit in a petri dish overnight. It was then rinsed three time with distilled water to remove any residual unbonded peptide.

Each of samples 1 through 11 were retained for about a week and then rinsed with 90% 2-propanol to sterilize, followed by drying in an oven at 120° Centigrade for 30 minutes to ensure that no trace propanol remained prior to inoculation with cells. The geometrical surface area of the sample coupons was 7.065 cm^{2.}

The positive control for alamarBlue® assay were tissue culture treated wells (9.6 cm²) seeded at the same density as the samples.

Cell Proliferation measured using the AlamarBlue® Assay: Saos-2 cells (ATCC HTB-85) grown in McCoy's 5A Media (ATCC 30-2007) supplemented with 15% FBS, were seeded at a density of 1.35*10⁵ onto the control (Sample 1) and the various treatment arms and positive control as enlisted above. All of the samples were bathed in 4 ml of media. On day 3, all the implants and positive control were removed from their wells and incubated in fresh 6 well plates and were trypsinized to detach the cells that were attached to the implants. After detachment, the cells in trypsin were centrifuged at 300 g for 5 mins and were resuspended in 2 ml of fresh cell media after removal of the trypsin. AlamarBlue® assay was performed by adding 200 ul of AlamarBlue® dye to the samples (10% of cell media volume, 1:10 ratio of dye: media) and fluorescence was allowed to develop for 24 hrs. After 24 hrs, the fluorescence of the individual samples and controls were measured at 590 nm (emission) after a 560 nm (excitation) resp. Unknown samples were converted to cell numbers using a known cell number standard curve using a generated linear or quadratic regression equation as shown in FIG. 1.

The AlamarBlue® Assay is designed to measure quantitatively the proliferation of various human cell lines, bacteria and fungi. The alamarBlue® Assay incorporates a fluorometric/colorimetric growth indicator based on detection of metabolic activity.

The assay is simple to perform since the indicator is water soluble, thus eliminating the washing/fixing and extraction steps required in other commonly used cell proliferation assays. activity. Specifically, the system incorporates an oxidation-reduction (REDOX) indicator that both fluoresces and changes color in response to chemical reduction of growth medium resulting from cell growth. The specific (fluorometric/colorimetric) REDOX indicator incorporated into alamarBlue® has been carefully selected because of several properties. First, the REDOX indicator exhibits both fluorescence and colorimetric change in the appropriate oxidation-reduction range relating to cellular metabolic reduction. Second, the REDOX indicator is demonstrated to be minimally toxic to living cells. Third, the REDOX indicator produces a clear, stable distinct change which is easy to interpret.

### Results

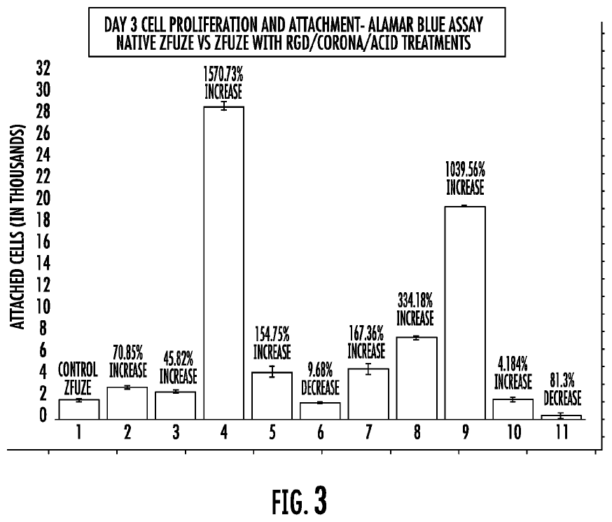

It is clear from the results as shown in FIG. 3 (percent increase or decrease in cellular attachment is shown in the graph below) that the treatments (arms) six, ten and eleven were not effective in enhancing surface attachment of cells relative to the control and showed either an insignificant increase in the case of sample 10 or a decrease of 9.68% and 81.3% in cellular attachment in the case of sample ten and eleven respectively. The most effective treatments were samples four, nine and eight which showed an exaggerated effect in increasing the cell response. Sample 4 demonstrated an astounding 1570% increase; sample 9 demonstrated a remarkable 1040% increase while sample 8 demonstrated a sizeable 334% increase in cell coverage over the control. All of the remaining samples exhibited statistically significant increase in cell attachment relative to control, which demonstrates substantial cause and effect in showcasing the efficacy of such treatments in enhancing the cell attachment characteristics of the PEEK-zeolite composite biomaterial.

Conclusion: The effect of the two most effective treatments: ammonia treatment, corona discharge and RGD peptide attachment onto PEEK-zeolite composite (Sample 4) and sulfuric acid treatment, corona discharge and RGD peptide attachment (Sample 9) was substantial in enhancing cellular attachment on the PEEK-zeolite composite.

While various aspects and embodiments have been disclosed herein, other aspects, embodiments, modifications and alterations will be apparent to those skilled in the art upon reading and understanding the preceding detailed description. The various aspects and embodiments disclosed herein are for purposes of illustration and are not intended to be limiting. It is intended that the present disclosure be construed as including all such aspects, embodiments, modifications and alterations insofar as they come within the scope of the appended claims or the equivalents thereof.

What is claimed is:

1. A cardiac replacement valve assembly comprising a valve body having a passageway through which blood flows when implanted into a host patient, a valve cooperating with said valve body to allow blood flow through said passageway in a first position and block blood flow through said passageway in a second position, and a suture ring extending from said valve body, wherein said valve body comprises a thermoplastic resin having ceramic particles incorporated therein, said ceramic particles being present in said resin in an amount sufficient to impart a negative charge to said exposed surface of said valve assembly.

2. The cardiac replacement valve of claim 1, wherein said suture ring comprising fabric.

3. The cardiac replacement valve of claim 2, wherein said fabric is infused with ceramic particles.

4. The cardiac replacement valve of claim 1, wherein said thermoplastic resin comprises polyetheretherketone.

5. The cardiac replacement valve of claim 1, wherein said ceramic particles comprise an aluminosilicate represented by the formula XM_{2/n}O·Al₂O₃·YsiO₂·ZH₂O wherein M represents an ion-exchangeable ion, n represents the atomic valency of the (metal) ion, X and Y represent coefficients of metal oxide and silica respectively, and Z represents the number of water of crystallization.

6. The cardiac replacement valve of claim 1, wherein said ceramic particles are a zeolite.

7. The cardiac replacement valve of claim 1, wherein said valve body has a surface region having been subjected to surface activation and comprising a therapeutically effective amount of an integrin-stimulating peptide immobilized thereon.

8. The cardiac replacement valve of claim 7, wherein said surface region has been activated by plasma treatment.

9. The cardiac replacement valve of claim 7, wherein said surface region has been activated by corona discharge treatment.

10. The cardiac replacement valve of claim 7, wherein said integrin-stimulating peptide is a peptide containing the amino acid sequence RGD.

11. The cardiac replacement valve of 7, said integrin-stimulating peptide is immobilized on said surface region with an activating agent providing functionality selected from the group consisting of amino functionality, hydroxyl functionality, amide functionality and carboxyl functionality.

12. The cardiac replacement valve of claim 1, wherein said ceramic particles comprise one or more metal ions.

13. A method of enhancing preferential polarization towards an M2 phenotype post-implantation of a cardiac valve, comprising excising from a patient a cardiac valve, and implanting in said patient a replacement cardiac valve assembly comprising a valve body having a passageway through which blood flows when implanted into said patient, a valve cooperating with said valve body to allow blood flow through said passageway in a first position and block blood flow through said passageway in a second position, and a suture ring extending from said valve body, wherein said valve body comprises a thermoplastic resin having ceramic particles incorporated therein, said ceramic being present in said resin in an amount sufficient to impart a negative charge to said exposed surface of said valve assembly.

14. The method of claim 13, wherein said ceramic particles comprise a zeolite.

15. The method of claim 13, wherein said thermoplastic resin comprises polyetheretherketone.

16. The method of claim 13, wherein said valve body has a surface region having been subjected to surface activation and comprising a therapeutically effective amount of an integrin-stimulating peptide immobilized thereon.

17. The method of claim 16, wherein said surface region has been activated by plasma treatment.

18. The method of claim 16, wherein said surface region has been activated by corona discharge treatment.

19. The method of claim 16, wherein said integrin-stimulating peptide is a peptide containing the amino acid sequence RGD.
